(19) United States
(12) Patent Application Publication
(10) Pub. No.: US 2004/0235801 A1
(43) Pub. Date: Nov. 25, 2004

(54) Therapy for stroke

(76) Inventors: Jean-Pierre Julien, Quebec (CA); Jasna Kriz, Quebec (CA)

Correspondence Address: OGILVY RENAULT, 1981 MCGILL COLLEGE AVENUE, SUITE 1600, MONTREAL, QC, H3A2Y3, CA

(21) Appl. No.: 10/788,478

(22) Filed: Mar. 1, 2004

Related U.S. Application Data
(63) Continuation-in-part of application No. 10/279,725, filed on Oct. 25, 2002, now abandoned.
(60) Provisional application No. 60/330,599, filed on Oct. 25, 2001.

(51) Int. Cl. A61K 31/65; A61K 31/455; A61K 31/426

(52) U.S. Cl. 514/152; 514/355; 514/365

(57) ABSTRACT

The present invention relates to a method for reducing symptoms related to stroke, this therapy comprising the administration of a therapeutically effective amount of at least two compounds selected from the group of an inhibitor of microglial activation, an antiglutaminergic agent and a voltage gated calcium channel blocker to a patient suffering from a neurodegenerative disease. The present invention also relates to a composition for reducing symptoms related to stroke, comprising a therapeutically effective amount of at least two compounds selected from the group of an inhibitor of microglial activation, an antiglutaminergic agent and a voltage gated calcium channel blocker in association with a pharmaceutically acceptable carrier.

This application is a continuation-in-part of the application Ser. No. 10/279,725 filed on Oct. 25, 2002.

### BACKGROUND OF THE INVENTION

(a) Field of the Invention

This invention relates a new therapy for the treatment of neurodegenerative diseases such as stroke, this new therapy delaying the onset of the disease and increasing the survival time of the subject.

(b) Description of Prior Art

With an incidence of approximately 250-400 in 100 000, stroke is the third leading cause of death in industrialized countries. Worldwide, about 5 million people died from stroke in 1999, which represents approximately 10% of all deaths (Dirnagl, et al., 1998, Trends Neurosci. 22, 391-397, Lo, E. H., et al., 2003, Nat. Rev. Neurosci. 4, 399-415). The chance of dying from initial stroke is 30-35%. However, for survivors there is a 35-40% risk of major disability. In practice, the term “stroke” refers to an umbrella of conditions caused by the occlusion or heamorrhage of blood vessel supplying the brain, with the blood flow being compromised within the territory of an occluded blood vessel. In all instances, stroke ultimately involves dysfunction and death of brain cells, and neurological deficits that reflect the location and size of the compromised brain area. Brain damage following transient or permanent ischemia results from a series of pathophysiological events that evolve in time and space (The National lnstituute of Neurological Disorders and Stroke rt-PA Group, 1995, New Eng. J. Med. 333, 1581-1587).

Early pathophysiological events following stroke are associated with specific time-window, starting few minutes after stroke and lasting several hours, including acute necrosis in the centre of infarction, followed by peri-infarct depolarizations, ionic dysbalance, excitotoxicity and oxidative stress (Dirnagl, et al., 1998, Trends Neurosci. 22, 391-397, Lo, E. H., et al., 2003, Nat. Rev. Neurosci. 4, 399-415). It is of importance that cerebral ischemic damages evolve at slower pace that previously believed. Neurons at the border of the ischemic territory can survive for many hours, even days after an ischemic insult (Dereski, M. O., et al., 1993, Acta Neuropathol. 85, 327-333). The delayed tissue response to brain ischemia includes inflammation and apoptosis (Dirnagl, U., et al., 1998, Trends Neurosci. 22, 391-397). To apply an efficient therapy for stroke it is crucial to understand the activation pattern and timing of different pathological pathways. At the present, a trombolysis using recombinant tissue plasminogen activator tPA is the only therapy approved for acute stroke by the US Food and Drug Administration. Because of the risk of hemorrhage that increases with time, treatment with tPA is limited to a 3-hour period immediately vascular occlusion (Hacke, W., et al., 1999, Neurology 53, S3-S15). A number of preclinical observations indicate that neuroprotective approaches might also confer beneficial effects. It reduces reperfusion injury and inhibits downstream targets in cell death cascade (Lo, E. H., et al., 2003, Nat. Rev. Neurosci. 4, 399-415). Considering that several pathways leading to a neuronal cell death are activated following cerebral ischemia, the rational approach may require the combination or addition of drugs in series that target distinct pathways during the evolution of ischemic injury.

It would be highly desirable to be provided with a new therapy delaying the onset of the disease and increasing survival time of a subject suffering from stroke.

### SUMMARY OF THE INVENTION

At the present there is no effective pharmacological treatment for stroke. The only currently approved therapy, trombolysis using recombinant tissue plasminogen activator is limited by three-hour period immediately after vascular occlusion as the risk of hemorrhage increases with the time. Many preclinical observations strongly suggest that neuroprotective approaches may also confer beneficial effects. Since many lines of evidence suggest that several pathological pathways leading to a cell death are activated in neurological disorders, simultaneously targeting different pathways should be a rational approach to treatment. It is provided herein evidence that three-drug cocktail consisting of minocyline, an antimicrobial agent with antiapoptotic and anti-inflammatory properties that blocks microglial activation, riluzole, a glutamate antagonist and nimodipine, a voltage gated calcium channel blocker, exerted remarkable neuroprotection in a mouse model of amyotrophic lateral sclerosis. Since excitoxicity, inflammation and altered calcium homeostasis are known to be associated with neuronal cell death following ischemia the same three-drug cocktail was tested in a mouse model of stroke (90 minutes middle cerebral artery occlusion followed by reperfusion). The therapy was administered 150 minutes after stroke and the effects were remarkable. The size of the infarction 24 and 72 hours after ischemia was decreased by more than 2-fold in treated animals. In addition, a battery of behavioral tests revealed significantly better clinical recovery of mice treated with the three-dug cocktail. These results indicate that this three-drug cocktail represents a novel and effective therapeutic approach for stroke in humans.

In accordance with the present invention there is provided a method for reducing symptoms related to stroke, the method comprising the administration of a therapeutically effective amount of at least two compounds selected from the group of an inhibitor of microglial activation, an antiglutominergic agent and a voltage gated calcium channel blocker to a patient suffering from said stroke.

In a preferred embodiment of the present invention, the compounds are administered simultaneously, but it is also understood that they could be administered consecutively as well.

In accordance with the present invention, there is provided a composition for reducing symptoms related to stroke, comprising a therapeutically effective amount of at least two compounds selected from the group of an inhibitor of microglial activation, an antiglutaminergic agent and a voltage gated calcium channel blocker in association with a pharmaceutically acceptable carrier.

In a preferred embodiment of the present invention, the inhibitor of microglial activation is minocycline, the antiglutaminergic agent is Riluzole and the voltage gated calcium channel blocker is Nimodipine.

All references cited in the present application are incorporated herein by reference.

### BRIEF DESCRIPTION OF THE DRAWINGS

FIG. 1A illustrates Miripine three-therapy increases life span of SOD1^{G37R} mice.

FIG. 1B illustrates Distribution of the mortality of miripine-treated vs control SOD1G37R mice according to their age in weeks;

FIG. 2 illustrates Miripine three-therapy improves muscle strength and delays disease onset; and

FIGS. 3A-3B are histograms showing the total number of axons in L4 and L5 ventral roots of normal mice (WT), drug-treated (tr) and control SOD1^{G37R} mice at the age of 10 months (A) and of 11 months (B);

FIGS. 4A-4O are micrographs showing the immunoreactivities of Cdk5, Cdk4 and activated capsase-3 in the spinal cord of WT mice (A, B, c), of drug-treated SOD1^{G37R} mice (D, E, F) and of control SOD1^{G37R} littermates (G, H, I) at 10 month-old as well as of drug-treated SOD1^{G37R} mice (J, K, L) and of control SOD1^{G37R} littermates (M, N, O) at 11 month-old;

FIGS. 5A-5C are micrographs illustrating the attenuation of microglial activation and astrogliosis in the spinal cord of SOD1^{G37R} mice; and

FIG. 6 compares brain sections stained with 2% tetrazolium red 24 hours after cerebral ischemia for control group and three-therapy group.

### DETAILED DESCRIPTION OF THE INVENTION

In accordance with the present invention, there is provided a new therapy useful for delaying the onset and increasing the survival time of a subject suffering from a neurodegenerative disease.

### EXAMPLE 1

### Three-Therapy for the Treatment of ALS

Material and Methods

Generation of SOD1^{G37R} Mice

Transgenic mice overexpressing SOD1G37R by˜5-fold (line 29) (3) were enriched in C57BL/6 background. Only mice heterozygous for the SOD1G37R transgene were used for our study. All mice were genotyped by Southern blotting. The use of animals and all surgical procedures were carried out according to The Guide of Care and Use of Experimental Animals of the Canadian Council of Animal Care.

Three-Therapy Treatment Protocol

The SOD1G37R mice were housed at the standard temperature (21° C.) and in light controlled environment with ad libitum access to the food and water. The study was carried out using transgenic littermates. The mouse littermates were fed a regular rodent food (Harlan, Teklad) and were randomly divided into Three Therapy-treated and control groups, including wild type littermates. At the age of 8-9 months, SOD1G37R mice from the experimental groups were administered triple medicated diet TD 01146 (Harlan, Teklad), containing 1000 mg/kg of minocycline, 500 mg/kg of riluzole and 500 mg/kg of nimodipine. All three compounds were purchased from (Sigma, Oakville,Canada). For the control groups the regular diet was continued until the mice reached end-stage disease. When progression of muscle weakness became marked, mice were fed at the bottom of their cages together with specially designed containers allowing them permanent access to water. Onset of the clinical disease was determined by measurement of motor strength, as described bellow and by the hind limb contraction when mice are suspended by their tail. At the end-stage disease, mice were monitored daily. They were killed when they started to lie on the side in their cages and when they start to express difficulties in grooming. To confirm the effects of combined three-therapy, two independent experiments were carried out on the different sets of trangenic SOD1G37R mice littermates. The therapy was applied at the late presymptomatic stage (7 and 8 months old mice) of disease.

Muscle Strength Test

The mice were allowed to grab vertically oriented wire (˜2 mm in diameter) with the loop at the lower end. The wire was designed in such a way that it allowed the mice to use both fore- and hind limbs. For more consistent measurements, the wire was maintained in the vertically oriented circular motion (circle radius ˜15 cm at 35 r.p.m.). Three tests (three consecutive days) were first used as a learning period trial in which all the mice learned to use both fore and hind limbs in order to stay longer on the circulating wire. Therefore, both skeletal muscle groups contribute in this strength assay. The maximum performance time was cut to 3 min. After the learning period, the test was performed once a week.

Immunohistochemistry

Mice were sacrified by intraperitoneal (I.p.) injection of chlorale hydrate, perfused with 16 g/l sodium cacodylate buffer (pH 7.4) followed by fixative (3% glutoraldehyde in sodium cacodylate buffer). Immunohistochemical studies were performed as previously described. Incubation with the primary antibodies anti-Cdk5 (C-8, 1:1000, Santa Cruz Biotechnology, Santa Cruz, Calif.), anti-glial fibrillary acidic protein monoclonal antibody (anti-GFAP, Sigma, Oakville, Canada, 1:200 dilution), anti-mouse-Mac2 rat monoclonal antibody (TIB-166) distributed by ATCC (Manassas, Va., 1:500 dilution), and anti p-p38, and anti cleaved caspase-3 rabbit polyclonal antibody (New England Biolab, Mississauga, Canada, 1:500 dilution) was performed overnight at room temperature in PBS/BSA. The labeling was developed using a vector ABC kit (Vector Laboratories, Burlington, Canada) and Sigma-fast tablets (Sigma, Oakville, Canada). Tissue section for the axonal counting were prepared for embedding in Epon as described previously.

Western Blots

The mice were sacrificed by overdose of chloralhydrate (i.p.). Immediately after, total protein extracts were obtained from L4-L5 spinal cord sections by homogenization in SDS-urea (0.5% SDS, 8M urea in 7.4 phosphate buffer) with a cocktail of protease inhibitors (PMSF 2 mM, Leupeptine 2 mg/ml, Pepstatin 1 mg/ml and Aprotinin). The protein was measured using a DC-protein assay™ (BioRad, Hercules, Calif.). The proteins were separated on 10% SDS-PAGE, transferred to nitrocellulose membranes and detected using monoclonal primary antibodies against anti-glial fibrillary acidic protein monoclonal antibody (anti-GFAP, Sigma, Oakville, Canada, 1:2000) anti-actin (C-4; 1:5000 Boehringer, Manheim) and anti p-p38, rabbit polyclonal antibody (anti p-p38, Thr 180/Tyr 182, New England Biolab, Mississauga, Canada 1:500 dilution). The Western blots were revealed using the Renaissance chemiluminescence kit (NEN Life Science, Boston, Mass.).

Data Analysis

Data are expressed as a mean±standard error. Statistical significance was assessed by two-tailed student t test (p≦0.05).

Results

Miripine Three-Therapy Increases the Life Span of SOD1^{G37R} Mice

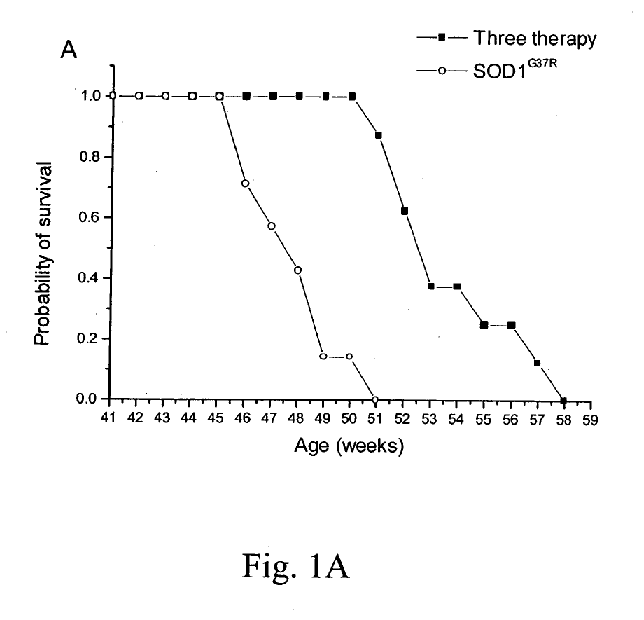

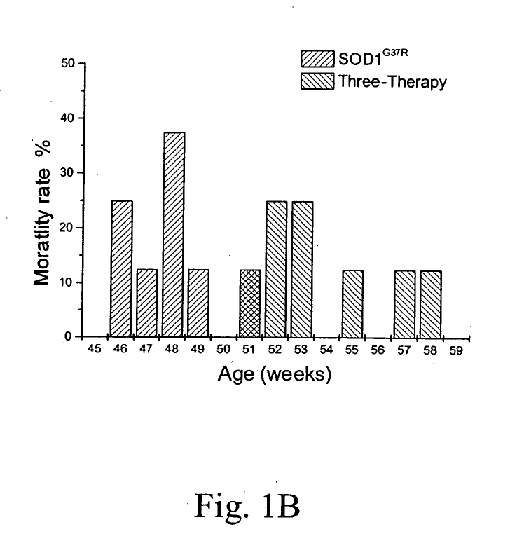

Mouse littermates heterozygous for the SOD1G37R transgene (line 29) were fed a regular rodent food (Harlan, Teklad). At late presymptomatic stage (8 and 9 months), the mice littermates were randomly divided into three-therapy treated and control groups. The three drugs (miripine) were delivered as a dietary supplement in the Special Custom Made Rodent Diet. FIG. 1A shows the survival curves of the miripine-treated (group A) and control SOD1G37R transgenic mice fed on regular diet. In FIG. 1A, the survival probability of transgenic mice is plotted as a function of their age in weeks. It shows that treatment with miripine starting at late presymptomatic stage of disease increased the average life span of SOD1^{G37R} mice by ˜6 weeks. When applied at the late presymtomatic stage, the miripine three-therapy increased longevity of SOD1^{G37R} mice by 7 weeks. As compared to the non-treated littermates, the average life span of miripine-treated SOD1^{G37R} mice was increased by 6 weeks (54.1±0.9; n=10 vs 48.0±0.6; n=10) (Table 1). Remarkably, even when applied after the onset of paralysis, in one group of the animals, the miripine three-therapy slowed down the progression of disease and delayed mortality by 4 weeks.

TABLE 1
Three-therapy delays the onset of disease and
increase longevity of SOD1^{G37R} mice

| | Muscle | Onset of | End Stage |
|---|---|---|---|
| | Weakness | Paralysis | Paralysis |
| SOD1^{G37R} mice | (weeks) | (weeks) | (weeks) |
| Three-Therapy | 47.8 ± 0.95* | 49.6 ± 1.06* | 54.1 ± 0.98* |
| Control | 43.0 ± 0.92 | 45.4 ± 0.59 | 48.0 ± 0.62 |

Values (Mean ± SEM), represents age of the mice expressed in weeks at the time of onset of the different stages of disease.
*significantly different from control (p ≦ 0.05). For both groups n = 10.

As shown in the FIG. 1B, the distribution of mortality rate for the tested mice revealed almost no overlapping between the two tested groups. For non-treated SOD1^{G37R} mice the peak of mortality was at 47-48 weeks, while the morality rate for the treated mice showed more equally spread distribution between 52 and 58 weeks. The difference in life span for some of the miripine-treated vs non-treated animals was more than 10 weeks (FIG. 1B).

Miripine Three-Therapy Delays the Onset of Disease and Muscle Strength Decline in SOD1^{G37R} Mice

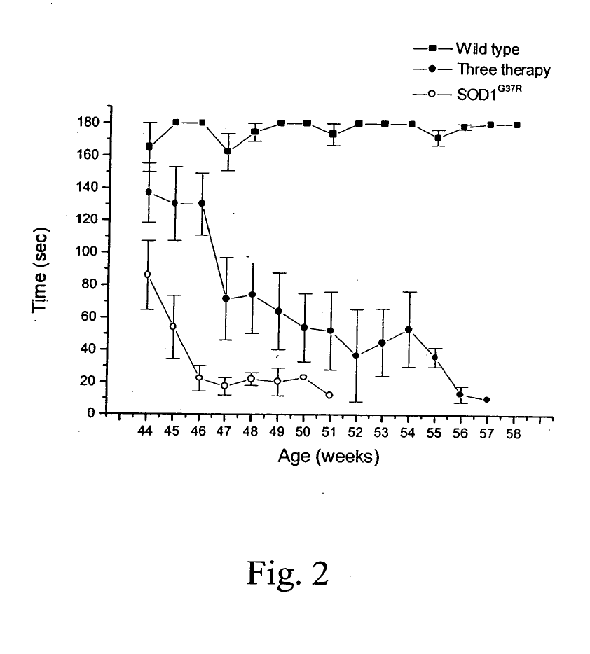

To determine the effects of miripine three-therapy on disease onset and progression in SOD1^{G37R} mice, a muscle strength assay was conducted (see Material and Methods). This assay is based on the time that single mouse was able to grip a vertical circulating wire. The results of the test revealed several different aspects of muscle strength changes associated with different stages of the disease progression. Unlike normal mice, the treated and control SOD1^{G37R} mice showed an age-dependent decline in hanging time (FIG. 2). In FIG. 2, unlike normal mouse littermates, measurement of muscle strength revealed an age-dependent decline in motor performance of SOD1^{G37R} mice. Treatment with miripine three-therapy prevented the decline in muscle strength and significantly improved motor performance of SOD1^{G37R} littermates until end-stage of disease. Muscle strength was indirectly measured as time that mice were able to send hanging on the circulating wire. Each point represents mean±SEM, * significant difference in comparison of miripine-treated vs non-treated SOD1^{G37R} mice, (p≦0.05 by two-tailed t test). The number of animals in each groups were for wild type, n=6; miripine-treated SOD1^{G37R} mice, n=8; control SOD1^{G37R} mice, n=8. The onset of disease in SOD1^{G37R} mice was characterized by a rapid decline in muscle strength (at the age of 43 to 44 weeks), followed by a slower declining stage of muscle strength (46 to 47 weeks of age) progressing to a stage of complete hind limb paralysis. Treatment with miripine three-therapy significantly delayed the first appearance of muscle weakness and significantly improved the motor performance of the treated SOD1^{G37R} mice throughout the tested period (FIG. 2). Applied three-therapy also significantly delayed the onset and slowed down the progression of the disease (FIG. 2 and Table 1).

Effective protection against the loss of motor axons in SOD1^{G37R} mice

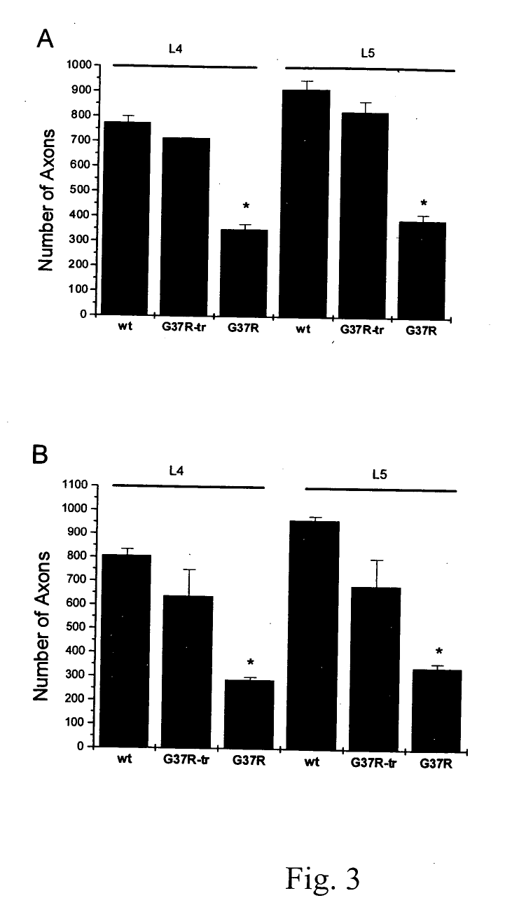

To assess whether the three-drug therapy delayed degeneration of motor neurons, the total number of motor axons in L4 and L5 ventral roots from treated SOD1^{G37R} mice (n=3 or 4) and control SOD1^{G37R} littermates (n=4) at early symptomatic stage of disease (44 weeks) and at late stage of disease (48 weeks) was counted as shown in FIGS. 3A and 3B. At early stage of disease, motor axons from treated SOD1G37R mice were mostly spared unlike axons from control SOD1^{G37R} littermates (FIG. 3A). For instance, at 44 week-old, the L5 ventral roots from control SOD1^{G37R} mice had 390±23 remaining axons whereas those from drug-treated mice had 823±41 axons, which is not significantly different from control values (911±36). A similar pattern was observed at the level of L4 ventral roots (713±20 for drug-treated vs. 352±20 for control SOD1^{G37R} mice). At 48 week-old, the number of remaining axons in the L4 and L5 ventral roots from drug-treated SOD1^{G37R} mice were of 637±112 and 685±115, respectively. Thus, while some axonal loss was evident at 48 weeks, the majority of motor axons were still present in the drug-treated SOD1^{G37R} mice.

Reduced Cdk5 Mislocalization and Capsasa-3 Activation

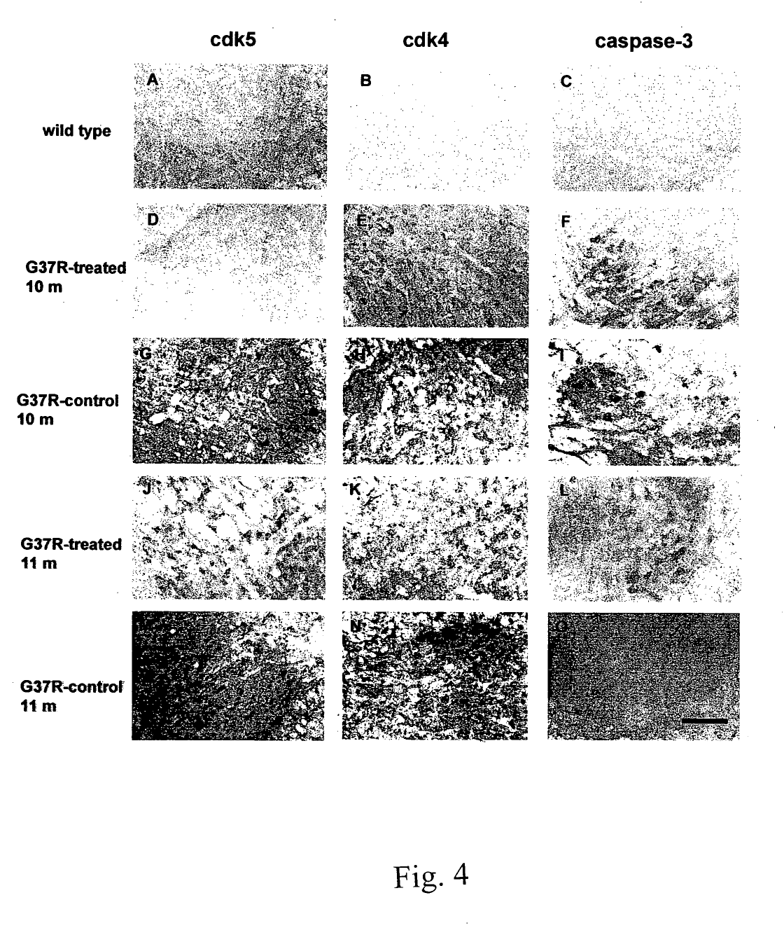

Recent studies demonstrated the involvement of caspase-3 activation in ALS pathogenesis. Activation of caspase-3 occurs late in the course of disease and it is associated with the loss of large motor neurons. Two other pathological hallmarks of degenerating neurons in SOD1^{G37R} mice are the mislocalization of Cdk5³⁰ and the nuclear localization of Cdk4³¹. Cdk5 is normally targeted to the cell membrane by its activator p35. However, in SOD1^{G37R} mice, Cdk5 is mostly detected in the cytoplasm of motor neurons. To examine whether the three-drug therapy attenuated the signals for markets of neurodegeneration in the spinal cord sections of SOD1^{G37R} mice, immunohistochemistry with anti-Cdk5, anti-Cdk4 and anti-caspase-3 antibodies were carried out.

Whereas the spinal motor neurons of control SOD1^{G37R} mice exhibited robust immunoreactivities for Cdk5 and Cdk4 at 10 month-old (FIGS. 4C and 4H), very low immunoreactivities were detected for Cdk5 and Cdk4 in spinal cord sections of 10 month-old SOD1^{G37R} mice under drug treatment (FIGS. 4D and 4E). At the age of 11 months, immunoreactivities for Cdk5 and Cdk4 were detected in spinal motor neurons of drug-treated SOD1^{G37R} mice but at reduced levels as compared to control SOD1^{G37R} mice (FIGS. 4J, 4K, 4M and 4N).

Antibodies against activated form of caspase-3 yielded a weak cytoplasmic immunostaining in several spinal motor neurons of drug-treated SOD1^{G37R} mice at 10 month-old (FIG. 4F), indicating that caspase-3 activation preceded axonal degeneration. Again, much stronger caspase-3 immunoreactivity was detected in moto neurons of 10 month-old control SOD1^{G37R} littermates. This shows that the beneficial effects of the three-drug treatment are associated with reduced signals for markers of neurodegeneration.

Three-Drug Therapy Attenuates Astrocytosis and Microglial Activation

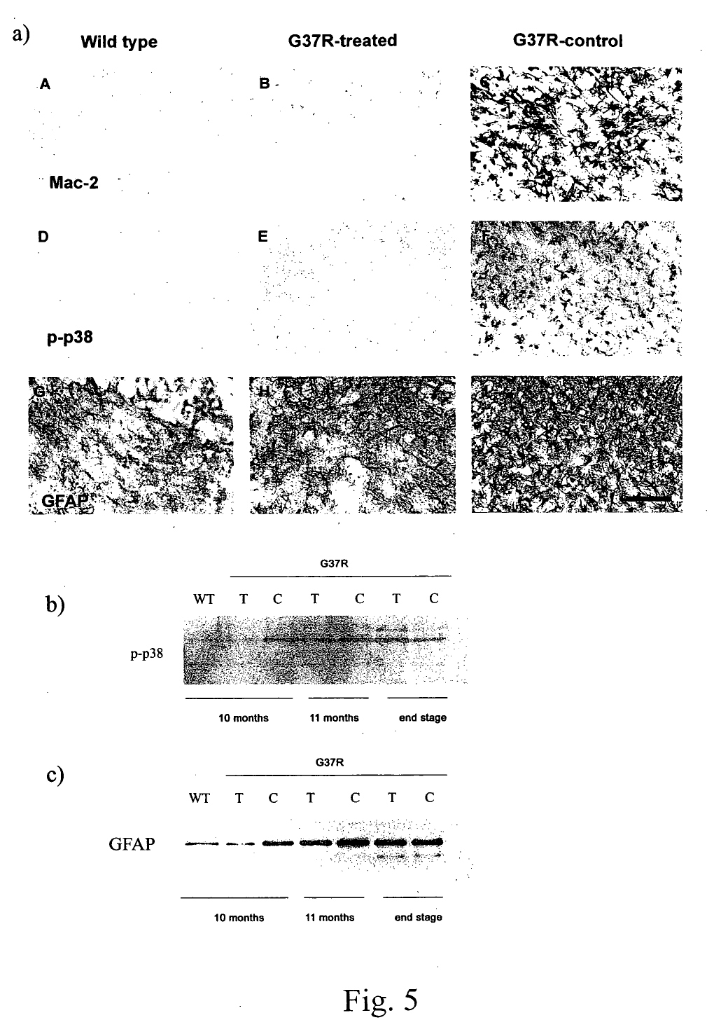

Astrocytosis and microgliosis are non-neuronal events that are likely to contribute to the neurodegenerative processes in ALS. Recently, it was shown that minocycline alone attenuates microglial activation but not astrocytosis in SOD1^{G37R} mice. To determine whether inclusion of nimodipine and riluzole together with minocycline exerted additional effects on glial cell activation in SOD1^{G37R}, immunohistochemistry and western blotting expression of Mac-2 and phosphorylated form p38 MAPK (p-p38) which are markers of microglial activation, and GFAP as a marker of astrogliosis were examined. At early symptomatic stage (44 weeks), the spinal cord sections of age-matched normal mice and drug-related SOD1^{G37R} mice were almost completely devoid of Mac-2 immunoreactivity (FIG. 5). In contrast, the spinal cord of control SOD1^{G37R} mice showed a robust Mac-2 immunoreactivity. The Mac-2 immunoreactive cells revealed morphology typical of activated microglia/macrophages (irregular shape, short processes) (FIG. 5A, panel C). A similar pattern of immunoreactivity was observed with antibodies against p-p38. Control SOD1^{G37R} mice yielded a strong p-p38 immunoreactivity in the white and gray matter (predominantly ventral horns) of the spinal cord (FIG. 5A, panel F). The p-p38 signal was considerably attenuated by the three-drug treatment. At the age of 44 weeks, the p-p38 immunoreactivity in the spinal cord of drug-treated SOD1^{G37R} mice was a low as in normal mice (FIG. 5a, panels D and E). this was further confirmed by western blotting. At age of 10 months weeks, spinal cord extracts from normal mice or from drug-treated SOD1^{G37R} mice (FIG. 5B). During disease progression, the levels of p-p38 in spinal cord extracts gradually increased in drug-treated SOD1G37R mice.

Unlike minocycline alone, the presence of riluzole and nimodipine in the three-drug therapy of the present invention markedly attenuated GFAP immunoreactivity in spinal cord sections of SOD1^{G37R} mice at 44 week-old (FIG. 5A, panels G and H). This was further confirmed by the weak GFAP immunostaining on western blot of spinal cord extracts from drug-treated SOD1^{G37R} mice as compared to control SOD1^{G37R} littermates (FIG. 5C).

Discussion

It is reported here for the first time a three-therapy pharmacological approach (combination of minocycline, riluzole and nimodipine) which is effective in delaying the onset of disease and mortality in a mouse model of ALS. Starting at the late presymptomatic stage (8 or 9 months of age) administration of miripine three-therapy in the diet significantly delayed the onset of motor neuron degeneration, attenuated astrogliosis and microglial activation, slowed down the disease progression and increased the motor performance of SOD1^{G37R} mice. This three-therapy approach delayed the onset of disease and increased the average longevity of ALS mice by 6 weeks. Moreover, for some mice the increase in life span exceeded 10 weeks.

Minocycline is a semisynthetic tetracycline derivative that effectively crosses blood-brain barrier and it is extensively used in human with relatively little side effects. It has been suggested that minocycline exerts neuroprotective effects by preventing microglial activation, reducing the induction of caspase-1 thereby decreasing the level of mature proinflammatory cytokine IL-1β and inhibiting cytochrome-c release from mitochondria, (Yrjätnheikki, J., et al. (1998) Proc. Natl. Acad. Sci. USA 95, 15769-15774; Yrjänheikki, J., et al. (1999) Proc. Natl. Acad. Sci. USA 96, 13496-13500; Chen, M., et al. (2000) Nat. Med. 6, 797-801, Zhu et al. (2002) Nature 417, 74-78). In addition, it has been shown that minocycline, doxycycline and their non-antibiotic derivatives (chemically modified tetracyclines) inhibit matrix metalloproteases, nitric oxide synthases, protein tyrosine nitration, cyclooxygenase-2 and prostaglandine E2 production. Recent studies performed with primary neurons and purified microglial cultures demonstrated that minocycline may also confer neuroprotection through inhibition of excitotoxin-induced microglial activation. Minocycline inhibits glutamate- and kainate-induced activation of p38 MAPK, exclusively activated in microglia.

A protection mechanism based on attenuation of microglial activation is compatible with an inflammation involvement in the pathology of neurodegenerative disorders. In human ALS, reactive microglia and reactive astrocytes are abundant in affected areas. Such gliosis as a phenomenon occurs also in the SOD1^{G37R} mouse model described here. It is known that minocycline, as a single therapy, slowed down progression of disease in SOD1^{G37R} mice but without affecting the onset. This shows that activated microglia, through the release of pro-inflammatory molecules, are more likely to play an active role in later stages of disease, contributing more to spreading of the neurodegenerative process (Julien, J. -P. (2001) Cell 104, 581-591).

Riluzole, a glutamate antagonist, is the only drug currently approved for therapy of ALS with only marginal effects on survival (Rowland, L. P. & Shneider, N. A. (2001) N. Eng. J. Med. 344, 1688-1699). In two controlled clinical trials it increased survival of ALS patients by 3-6 months. Although the precise mechanism of action of riluzole has not been fully elucidated, it appears to involve interference with excitatory amino acid (EAA) in the CNS, possibly through inhibition of glutamic acid release, blockade or inactivation of sodium channels and/or activation of G-protein coupled transduction pathways. When tested as a single therapy in SOD1 mutant mice it increased survival for 13-15 days without affecting the onset of disease (Gurney, M. E., et al. (1996) Ann. Neurol. 39,147-157).

At the present, there is enough substantial evidence supporting hypothesis that Ca2+ influx through L-type voltage gated channels may contribute to neuronal death. Recent study by demonstrated that Ca2+ entry through L-type calcium channels induces mitochondrial disruption and cell death. In addition, antibodies against voltage gated calcium channels have been isolated from cerebrospinal liquor of some ALS patients, and when tested in in vitro and in vivo condition they induced selective increase of intracellular calcium in motor neurons associated with cell injury and death. Calcium channel blocking agents antagonize EAA receptor and decrease calcium entry into damaged neurons that may slow down or reverse neurodegenerative processes in ALS.

Nimodipine is the L-type voltage gated calcium channel blocker with preferential effects on CNS (Langley, M. S. & Sorkin, E. M. (1989) Drugs 37, 669-699). It exerts anxiolytic and antiamnestic effect in animals, it facilitates learning in old animals, exhibits certain neuroprotective effects in ischemia/hypoxia induced nerve damage, posses some anticonvulsant properties. Recently, it has been shown that nimodipine promotes regeneration and functional recovery after intracranial facial nerve crush. However, tested as a mono-therapy in one controlled clinical trial nimodipine was not effective in slowing down the disease progression in ALS patients.

Previous studies indicated that nimodipine and riluzole when applied in human ALS or mouse model as a single therapy exert very modest or no effects on ALS. In contrast, minocycline was quite effective in slowing down the disease progression in mouse model of ALS. However, combination of minocyline, riluzole and nimodipine, applied as a three-therapy increased average life span of SOD1 G37R mice by 6 weeks (FIG. 1, Table 1), which represents 100% increase in efficacy as compared to minocycline therapy. Remarkably, for some of the animals difference in the life span was more than 10 weeks (see FIG. 2). As shown in the FIG. 3 and Table 1, treatment with three-therapy also significantly delayed decline of the muscle strength and disease onset of SOD1G37R mice.

To date, aside from miripine three-therapy, no pharmacological treatment was able to delay both, the onset and the progression of disease in a mouse model of ALS. Comparing the effectiveness of our three-therapy approach to a pharmacological efficacy of every single compound contained in our drug cocktail, it is evident that they acted in synergy. Multiple factors and pathological pathways, that are not mutually exclusive, are involved in the pathogenesis of ALS and the disease progression. Our results clearly demonstrated that strategic and simultaneous pharmacological intervention on three different pathological pathways, riluzole as antiglutaminergic agent prevents excitotoxic effects of glutamate; nimodipine as voltage gated calcium channel blocker prevents excessive calcium influx into depolarized damaged neurons and minocycline, as a inhibitor of microglial activation, prevents toxic effects of activated microglia, resulted in remarkably effective treatment.

### EXAMPLE 2

### Three-Therapy for the Treatment of Stroke

Material and methods

Experimental animals

Adult (3-5 months old) male C57BL/6 mice were obtained from Charles River Inc. (Saint-Constant, Quebec, Canada). All experimental procedures were approved by the MUHC animal care committee according to the guidelines of the Canadian Council for Animal Care. All animals were allowed free access to water before and after surgery.

Surgical Procedures

Unilateral focal cerebral ischemia was induced by occlusion of the medial cerebral artery (MCA) in the left cerebral hemisphere during 1.5 hours. The MCA occlusions in mice is carried out on ˜30 C57BI/6 mice weighing 20-25 g from an adaptation of a method previously described in rats (Beaulieu, J. M., Kriz, J. and Julien, J. P. ,2002, Brain.Res. 946, 153-161). The animals were anesthetized by administration of ketamine/xylazine mg/kg i.m. To avoid hypothermia, the mouse body temperature was maintained at 37° C. with an infrared heating lamp and warm pad. The MCA occlusion was induced by the intraluminal occlusion method using a silicone-coated nylon monofilament 6-0 thread. Under operating microscope, the left common carotid artery and ipsilateral external carotid artery (ECA) was exposed through a midline neck incision and was carefully isolated from surrounding tissue. The occipital artery branches of the ECA were then be isolated, dissected and coagulated. The ECA was dissected further distally and coagulated. The internal carotid artery (ICA) was isolated and carefully separated from the adjacent vagus nerve. The pterygopalatine artery was ligated close to its origin with 6-0 silk suture. Then, the 6-0 silk suture was tied loosely around the immobilized ECA stump, and a 14 mm length of 6-0 monofilament nylon suture was inserted via the proximal ECA into the ICA and then into the circle of Willis occluding the MCA. The silk suture around the ECA stump was tighten around the intraluminal suture to prevent bleeding. 90 minutes after intraluminal inclusion, the intraluminal suture was carefully removed. The neck incision was closed with silk sutures and the mice were allowed to survive for 24-72 hours following reperfusion.

Treatment Protocol

Three-therapy treatment was first administered 150 minutes after 90 minutes of MCA occlusion. In all experiments, non-treated control mice were injected with saline (i.p.) whereas treated mice were administered either minocycline (70 mg/kg i.p. per day) or a three-therapy (minocycline 70 mg/kg, riluzole 20 mg/kg of and nimodipine 10 mg/kg i.p) 1× per day during 72 hours period. All three compounds were purchased from (Sigma, Oakville,Canada). After 24 or 72 hours following surgery, the mice were sacrificed, and the brains quickly removed in order to proceed with the evaluation of the infarction size.

Size of Infarction

The area of infarct was estimated in the minimum of animals (5-7 mice) from each experimental group. The mice were sacrificed by overdose of anesthetic, the brains were quickly removed, placed in a special mouse brain mold, and cut into 1 mm coronal sections. Brain sections were than immersed into a 2% solution of 2, 3, 5 tryphenyltetrazolium or tetrazolium red. The area of infarct was estimated according to the tetrazolium red staining, calculated in arbitrary units pixels and expressed as a percentage of a total control area, the contralateral non-stroked side of the brain. The infarct area represents the area of 6 consecutive 1-mm coronal sections affected with MCA occlusion. The infarct volume was calculated by summing up the infarct area in multiple slices. The size of the brain infarct in mice treated with the three-therapy versus minocycline alone and non-treated mice were compared. The analysis was performed by using Scion Image™ processing and analysis program.

Behavioral Analysis

To examine the effects of the three-therapy on the clinical recovery of mice after cerebral ischemia, the behavior of the mice and neurological deficits were investigated using modified SHIRPA protocol (Derek, C. R., et al., 1997, Mammalian Genome 8, 711-713; Derek, C. R., et al., 1999, Behav. Brain Res. 105, 207-217). Each animal was given a functional score that was reflecting the extent of neurological deficit. The animals were monitored during the period of postoperative recovery staring at 24 hours after stroke to the period of 72 hours operation. The functional scores were compared between the three-therapy treated and non-treated mice.

Results

Three-drug Cocktail Markedly Decreases the Size of Infarction Induced by Transient MCA Occlusion

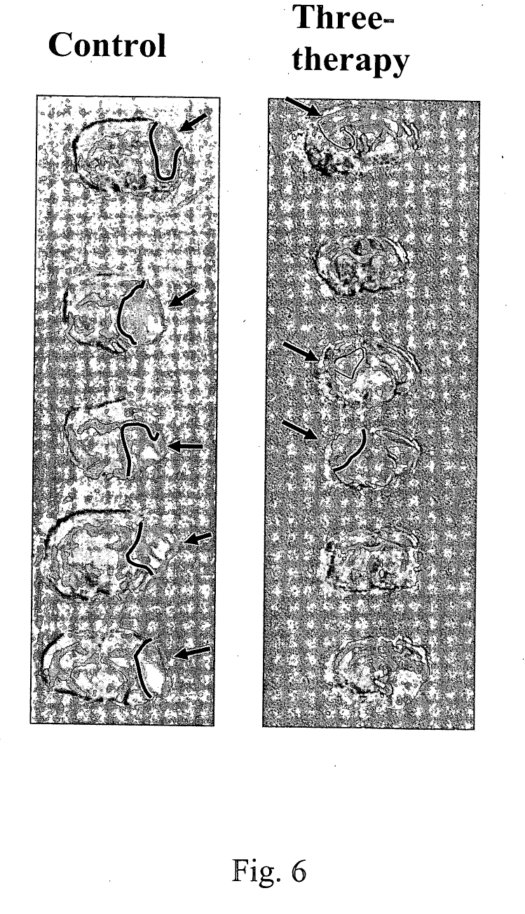

Unilateral focal cerebral ischemia was induced by MCA occlusion in the left hemisphere for 90 minutes followed by 24 or 72 hours reperfusion period (Beaulieu, J. M., Kriz, J. and Julien, J. P. ,2002, Brain.Res. 946, 153-161). Staining of ischemic brains with tetrazolium red confirmed that unilateral MCA provoked localized ischemic damage in the one cerebral hemisphere that included the hippocampus, the thalamus, striatum and parts of the adjacent cerebral cortex. As tetrazolium red is a vital die, necrotic part of the tissue is not stained and stays white. Note that this procedure completely spared the contralateral hemisphere that is used for internal control (see FIG. 6). As shown in the FIG. 6, comparison of the treated and non-treated animals revealed that, treatment with the three-drug cocktail when applied 150 minutes after stroke and 60 minutes after reperfusin markedly reduced the size of the necrotic-infracted area.

To further analyze and quantify the changes in the size of the infarct area, for each animal 6 consecutive 1 mm section affected with MCA occlusion were stained with tetrazolium red, analyzed, and volume of the infarction was calculated by summing up the infarct area in the multiple slices. The infarct area is than expressed as a percentage of contralateral (non-ischemic) part of the brain. Quantitative analysis of the infarct area 24 hours after MCA occlusion revealed that three-therapy treatment applied 150 minutes after stroke decreased the size of the ischemic lesion by 2-fold, as compered with non-treated animals. Treatment with minocycline alone was less efficient. (Table 2). As shown in Table 2, three-therapy decreases the size of the ischemic lesion 24 hours after 90 minutes middle cerebral artery occlusion (MCAO), followed by 24 hours reperfusion period. Three-therapy was administered 2.5 hours after MCAO and 1 hour after reperfusion. The extent (area) of the ischemic lesion was measured using tetrazolium red vital staining and expressed as a percentage of the contralateral (non-ischemic) side of the brain. To investigate whether an increase in treatment duration will have some additional neuroprotective effects, we applied three-therapy treatment protocol during 72 hours time period. As shown in Table 3, 3 days three-therapy treatment decreased the size of infarction by more than 2-fold when compared with non-treated mice, suggesting that longer therapy may have some additional beneficial effects.

TABLE 2
Comparison of % of ischemic area for control,
three-therapy and minocycline treatments

| | Control | Three-therapy | Minocycline |
|---|---|---|---|
| | (n = 10) | (n = 6) | (n = 3) |
| % of ischemic area | 49.6% | 23.9% | 38.4% |

TABLE 3
Comparison of % of ischemic area for control
and three-therapy treatment

| | Control | Three-therapy |
|---|---|---|
| | (n = 5) | (n = 5) |
| % of ischemic area | 48.5% | 19.4% |

Three-Therapy Treatment Increases Clinical Recovery of Mice After MCA Occlusion

Since neurological deficits and physical disabilities are the major problem of stroke survivors, it was of great interest to investigate the effects of three-therapy on clinical recovery of mice. The behavior of mice was investigated using the modified SHIRPA protocol, an established method to monitor mice behavior and evaluate neurological deficits (Derek et al., 1997, 1999). The experimental animals were monitored 24, 48 and 72 hours following stroke. Each animal was given a functional score that reflected the extent of neurological deficits, and the functional scores were than compared between the animals that underwent three-therapy treatment and non-treated animals. As shown in the Table 4, the three-therapy approach of the present invention significantly improved clinical recovery of the mice. A battery of test used in this examination revealed that non-treated animals on average exert less spontaneous activity, body position was flat, respiration rate was more irregular and circulating behavior, as a sign of developed cerebral lesion was more pronounced and persisted in non-treated mice. In addition, non-treated mice had lower scores for righting reflex. The only task in which performance of three-therapy treated mice was less efficient is the “wire maneuver grip test”. This is presumably due to the pharmacological effects of nimodipine that can in part induce slight muscle relaxation.

TABLE 4
Behavioral tests results from three-therapy treated and non-treated
mice and 24, 48 and 72 hours following MCAO

| | | | 24 h | 24 h | 48 h | 48 h | 72 h | 72 h |
|---|---|---|---|---|---|---|---|---|
| | Score | Ranger of scores | Control | Three-therapy | Control | Three-therapy | Control | Three-therapy |
| Body position | 0-5 | Completely float to | 2.4 ± 0.3 | 2.8 ± 0.1 | 3.0 ± 0.4 | 3.75 ± 0.2 | 3.1 ± 0.5 | 4.3 ± 0.4 |
| | | repeated leaping | | | | | | |
| Spontaneous | 0-4 | None to repeated | 1.9 ± 0.3 | 2.7 ± 0.1 | 2.5 ± 0.3 | 2.9 ± 0.1 | 2.75 ± 0.4 | 3.25 ± 0.2 |
| activity | | vigorous | | | | | | |
| | | movement | | | | | | |
| Respiration rate | 0-3 | Regular to | 1.16 ± 0.5 | 0.4 ± 0.2 | 0.5 ± 0.5 | 0 | 0.5 ± 0.2 | 0 |
| | | hyperventilation | | | | | | |
| Tremor | 0-2 | None to marked | n | n | n | N | n | n |
| Circulating | 0-1 | Absent or present | 0.66 ± 0.2 | 0.8 ± 0.2 | 0.66 ± 0.2 | 0.25 ± 0.2 | 0.66 ± 0.2 | 0.25 ± 0.2 |
| behavior | | | | | | | | |
| Piloerection | 0-1 | Absent or present | 0.83 ± 0.1 | 0.6 ± 0.2 | 0.83 ± 0.6 | 0.5 ± 0.2 | 0.83 ± 0.6 | 0.25 ± 0.2 |
| Wire manoeuvre | 0-4 | Active and grips to | 2.8 ± 0.4 | 2.3 ± 0.3 | 2.3 ± 0.3 | 2 ± 0 | 2.5 ± 0.4 | 1.6 ± 0.2 |
| | | falls immediately | | | | | | |
| Tail suspension | 0-2 | Flexion or rotation | 1.16 ± 0.2 | 1 ± 0.0 | 1.17 ± 0.2 | 0.5 ± 0.2 | 1.16 ± 0.1 | 0.5 ± 0.2 |
| (flexion or | | | | | | | | |
| rotation) | | | | | | | | |
| Corneal reflex | 0-2 | Blink response to | 1.83 ± 0.16 | 1.9 ± 0.1 | 1.91 ± 0.1 | 2 ± 0.0 | 1.75 ± 0.1 | 2 ± 0.0 |
| | | light tactile | | | | | | |
| | | stimulus | | | | | | |
| Righting reflex | 0-3 | No impairment to | 2.5 ± 0.5 | 3 ± 0.0 | 2.66 ± 0.3 | 3 ± 0.0 | 2.5 ± 0.5 | 3 ± 0.0 |
| | | fails to right when | | | | | | |
| | | placed on back | | | | | | |

Discussion

Here, it is reported a novel and efficient neuroprotective pharmacological cocktail for treatment of stroke. This treatment is based on the combination of three drugs, minocycline, riluzole and nimodipine, aiming to distinct targets in the pathways to neuronal cell death following cerebral ischemia. Remarkably, when applied 150 minutes after the onset of ischemia, this three-therapy treatment decreased the size of infarction by 2-fold at 24 and 72 hours after stroke. In addition, such a therapeutic approach also induced better clinical recovery of mice subjected to middle cerebral artery occlusion (see Tables 2, 3 and 4).

Considering that several pathological pathways, including excitotoxicity, inflammation and altered calcium homeostasis are activated and involved in the processes associated with the neuronal cell death following cerebral ischemia, the rationale of this pharmacological approach was to simultaneously target these specific pathways in order to achieve efficient neuroprotection.

Minocycline is a semisynthetic tetracycline derivative that effectively crosses blood-brain barrier and it is extensively used in human with relatively little side effects (Goulden, V., et al., 1996, Br. J. Dermatol. 134, 693-695). It has been suggested that minocycline exerts neuroprotective effects by preventing microglial activation, reducing the induction of caspase-1 and inhibiting cytochrome-c release from mitochondria (Yrjänheikki, J., et al., 1998, Proc. Natl. Acad. Sci. USA 95, 15769-15774; Yrjätnheikki, J., et al., 1999, Proc. Natl. Acad. Sci. USA 96, 13496-13500; Chen, M., et al., 2000, Nat. Med. 6, 797-801, Zhu et al., 2002, Nature 417, 74-78). In addition, it has been shown that minocycline inhibits matrix metalloproteases, nitric oxide synthases, protein tyrosine nitration, cyclooxygenase-2, and prostaglandin E2 production and may also confer neuroprotection through inhibition of kainate-induced microglial activation (Golub, L. M. et al. ,1998, Adv.Dent.Res. 12, 12-26, Tikka, T et al., 2001, J. Neurosci. 21, 2580-2588). Minocycline exerted neuroprotective effects in the experimental models of cerebral ischemia, Huntington's and Parkinson's disease (Chen, M. et al., 2000, Nat. Med. 6, 797-801, Du, Y et al., 2001, Proc. Natl. Acad. Sci. U.S.A. 96, 14669-14674). Recently our group and others (Van Den Bosch, L. et al., 2002, NeuroReport 13, 1-4; Zhu, S. et al., 2002, Nature 417, 74-78) demonstrated beneficial effects of minocycline in the mouse models of ALS. A protection mechanism based on attenuation of microglial activation is compatible with an inflammation involvement in the pathology of neurodegenerative disorders including cerebral ischemia (Dirnagl, U., et al., 1998, Trends Neurosci. 22, 391-397, Julien, J. P., 2001, Cell 104, 581-591 and Lo, E. H., et al., 2003, Nat. Rev. Neurosci. 4, 399-415).

Riluzole is a glutamate antagonist whose precise mechanism of action has not been fully elucidated. It appears to involve interference with excitatory amino acids in the central nervous system, presumably through inhibition of the presynaptic release of gluatamate, blockade or inactivation of sodium channels and/or activation of G-protein coupled transduction pathways (Martin, D. et al., 1993, Eur. J. Pharmacol. 250, 473-476; Bensimon et al., 1994, N. Eng. J. Med. 330, 585-591; Gurney, M et al., 1996, Ann. Neurol. 39, 147-157).

Nimodipine is a L-type voltage-gated calcium channel blocker with the preferential effects on central nervous system (Singh, B. ,1986, Br. J. Pharmacol. 21, 109-121). In PC12 cells treatment with nimodipine prevented massive Ca2+ influx through voltage-gated calcium channels induced by membrane depolarization, a phenomenon associated with mitochondrial disruption and followed by cell apoptosis and/or necrosis (Cano-Abad, M. F. et al., 2001, J.Biol.Chem. 43, 39695-39704). It also exhibits a neuroprotective effect in hypoxia-ischemia-induced brain damage, and it posses certain anticonvulsant effects against kainic acid-induced seizures (Korenkov, A. I., et al., 2000, Neurosurg. Rev. 23, 145-150; Kriz, J. et al., 2003, Epilepsy Res.).

Many preclinical observations indicate that treatment of stroke is suboptimal without combining clot-lysing drugs with neuroprotective therapy. Such combination reduces reperfusion injury and inhibits downstream targets in cell death cascade. The three-drug cocktail described in the present application exerted remarkable neuroprotection and significantly better clinical recovery in a mouse model of stroke. Thus, the three-drug cocktail described in the present application represents a novel and efficient neuroprotective treatment for stroke in humans.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for reducing symptoms related to stroke, said therapy comprising the administration of a therapeutically effective amount of at least two compounds selected from the group of an inhibitor of microglial activation, an antiglutominergic agent and a voltage gated calcium channel blocker to a patient suffering from said stroke.

2. The method of claim 1, wherein said inhibitor of microglial activation is minocycline.

3. The method of claim 1, wherein said antiglutaminergic agent is Riluzole.

4. The method of claim 1, wherein said voltage gated calcium channel blocker is Nimodipine.

5. The method of claim 1, wherein said compounds are administered simultaneously.

6. The method of claim 1, wherein said compounds are administered consecutively.

7. A composition for reducing symptoms related to stroke, comprising a therapeutically effective amount of at least two compounds selected from the group of an inhibitor of microglial activation, an antiglutaminergic agent and a voltage gated calcium channel blocker in association with a pharmaceutically acceptable carrier.

8. The composition of claim 7, wherein said inhibitor of microglial activation is minocycline.

9. The composition of claim 7, wherein said antiglutaminergic agent is Riluzole.

10. The composition of claim 7, wherein said voltage gated calcium channel blocker is Nimodipine.
